(19) United States
(12) Patent Application Publication
(10) Pub. No.: US 2004/0171611 A1
(43) Pub. Date: Sep. 2, 2004

(54) Crystalline acetic acid solvate of meloxicam

(75) Inventors: Guenter Trummlitz, Warthausen (DE); Rainer Soyka, Biberach (DE); Peter Sieger, Mittelbiberach (DE); Ulrike Werthmann, Biberach (DE); Peter Luger, Berlin (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma GmbH & Co. KG, Ingelheim (DE)

(21) Appl. No.: 10/667,643

(22) Filed: Sep. 22, 2003

Related U.S. Application Data
(60) Provisional application No. 60/428,617, filed on Nov. 22, 2002.

(30) Foreign Application Priority Data
Sep. 30, 2002 (DE) DE 102 45 882.0

(51) Int. Cl. A61K 31/5415; C07D 417/02

(52) U.S. Cl. 514/226.5; 544/49

(57) ABSTRACT

The present invention relates to a new crystalline modification of the compound 4-hydroxy-2-methyl-N-(5-methyl-2-thiazolyl)-2H-1,2-benzothiazin-3-carboxamide-1,1-dioxide in the form of an acetic acid solvate of formula I as well as the use thereof as a pharmaceutical composition.

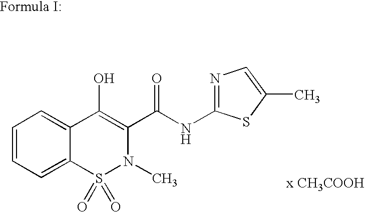

### RELATED APPLICATIONS

Benefit of U.S. Provisional Application Serial No. 60/428,617, filed on Nov. 22, 2002 is hereby claimed.

### FIELD OF THE INVENTION

The present invention relates to the new modification of the compound 4-hydroxy-2-methyl-N-(5-methyl-2-thiazolyl)-2H-1,2-benzothiazin-3-carboxamide-1,1-dioxide in the form of the crystalline acetic acid solvate of formula I as well as the use thereof as pharmaceutical compositions.

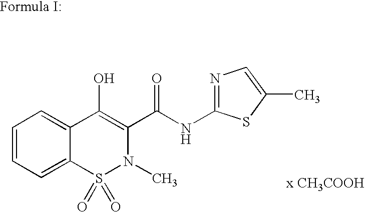

### BACKGROUND OF THE INVENTION

The compound 4-hydroxy-2-methyl-N-(5-methyl-2-thiazolyl)-2H-1,2-benzothiazin-3-carboxamide-1,1-dioxide(meloxicam) has already been described in European Patent Application EP 0 002 482 and as a non-steroidal anti-inflammatory active substance (NSAID) belongs structurally to the category of the acidic enolcarboxamides (oxicams). A corresponding preparation is on the market under the trade mark Mobic®. WO 99/49845 describes an oral suspension of the active substance while WO 99/49867 describes a tablet containing meloxicam-meglumine salt.

Depending on the pH and the solvents used, X-ray structural analysis shows that meloxicam crystallises out in four different prototropic forms: the anionic, the acid enolic, the zwitterionic and the cationic form (G. Trummlitz et al., Eur. J. Pharm. Sciences 1996, 4, 175-187).

Pharmaceutically acceptable meloxicam is obtained by crystallisation from nonpolar organic solvents in the enol form. Under physiological conditions (pH=7.4) the anionic (A) is the predominant form obtained. It is also accepted that under aqueous conditions zwitterionic forms (C) are additionally present. There are two possible prototropic zwitterionic forms, one of which (the amidate) may be resonance-stabilised. It has also been found that meloxicam in the form of the hydrogen sulphate is present as cation (B).

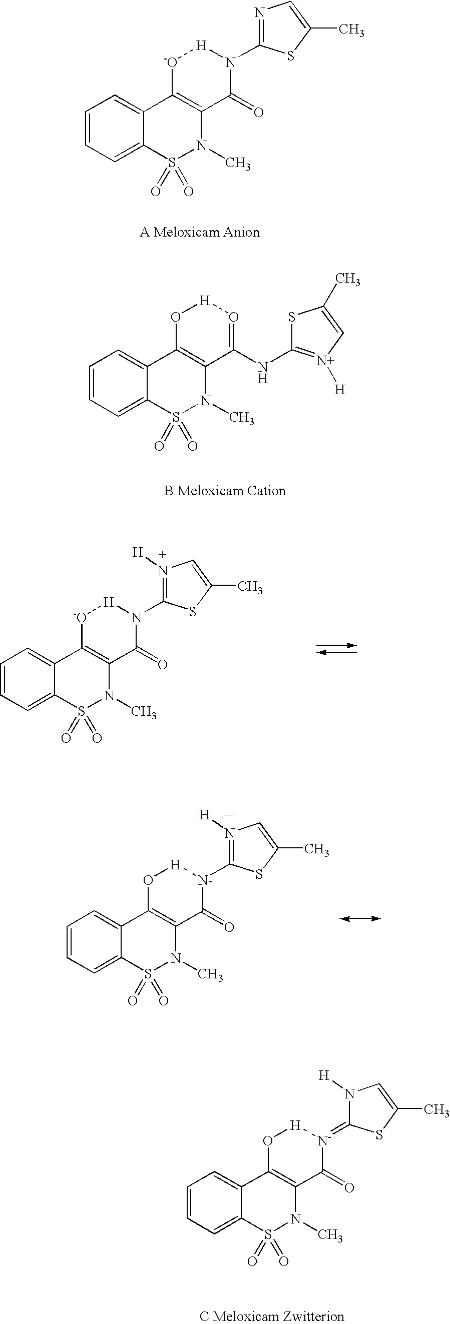

### BRIEF DESCRIPTION OF THE INVENTION

The aim of the present invention was to prepare another enol form of meloxicam suitable for pharmaceutical use.

The acetic acid solvate of the compound 4-hydroxy-2-methyl-N-(5-methyl-2-thiazolyl)-2H-1,2-benzothiazin-3-carboxamide-1,1-dioxide according to the invention, like pure meloxicam, has an antiphlogistic activity, inhibits the pain of inflammation and is suitable for treating rheumatic diseases.

The compound according to the invention is therefore suitable for treating all peracute, acute, subacute, chronic and recurring inflammation, particularly for treating the symptoms of acute episodes of intermittent or chronic activated arthrosis as well as for long-term symptomatic treatment of rheumatoid arthritis (chronic polyarthritis) and for the symptomatic treatment of ankylosing spondylitis (Bechterew's disease).

It has also been found that the compound of general formula I is suitable for the prevention and treatment of neoplasias which produce prostaglandins or secrete cyclooxygenase, including benign and cancerous tumours, growths and polyps. Neoplasias which (frequently) produce prostaglandins comprise for example malignant brain tumours, bone cancer, epithelial cell neoplasia such as basal cell carcinoma, adenocarcinoma, cancers of the gastrointestinal tract such as lip cancer, mouth cancer, oesophageal cancer, cancer of the small intestine and stomach cancer, large bowel cancer, liver cancer, bladder cancer, pancreatic cancer, ovarian cancer, cancer of the womb, lung cancer, breast cancer and skin cancer, prostate cancer, kidney cell carcinoma and other known types of cancer which affect the epithelial cells in the body.

The compound according to the invention is also suitable for treating acute pain, such as for example toothache after tooth extractions, post-traumatic and postoperative pain, headache, acute sciatica, acute back pain, tendonitis, cervicobrachial syndrome and tennis elbow as well as for the treatment of persistent pain, such as for example backache or pain caused by tumours.

The abovementioned pharmacologically valuable properties of the NSAIDs disclosed in the prior art are the prerequisite for effective use of the compounds as pharmaceutical compositions. However, an active substance must also satisfy other requirements in order to be permitted for use as a pharmaceutical composition. These parameters are to a great extent connected with the physicochemical nature of the active substance.

As the crystal modification of an active substance is important to the reproducible active substance content of a preparation, there is a need to clarify as far as possible any existing polymorphism of an active substance present in crystalline form. If there are different polymorphic modifications of an active substance care must be taken to ensure that the crystalline modification of the substance does not change in the pharmaceutical preparation later produced from it. Otherwise, this could have a harmful effect on the reproducible potency of the drug. Against this background, active substances characterised by only slight polymorphism are preferred.

Another criterion which may be of exceptional importance under certain circumstances depending on the choice of formulation or the choice of manufacturing process is the solubility of the active substance. If for example pharmaceutical solutions are prepared (e.g. for infuisions) it is essential that the active substance should be sufficiently soluble in physiologically acceptable solvents. It is also very important for drugs which are to be taken orally that the active substance should be sufficiently soluble. The solubility of an active substance is a further prerequisite for its absorption, as different modifications of an active substance have different solubilities and may thus differ in other physical/chemical properties.

The problem of the present invention is therefore to provide a pharmaceutically active substance which not only is characterised by high pharmacological potency but also satisfies the above-mentioned physicochemical requirements as far as possible.

### DETAILED DESCRIPTION OF THE INVENTION

Surprisingly, it has been found that the problem outlined above is solved by the crystalline acetic acid solvate of the compound 4-hydroxy-2-methyl-N-(5-methyl-2-thiazolyl)-2H-1,2-benzothiazin-3-carboxamide-1,1-dioxide of formula I.

A first object of the present invention is thus the crystalline acetic acid solvate of the compound 4-hydroxy-2-methyl-N-(5-methyl-2-thiazolyl)-2H-1,2-benzothiazin-3-carboxamide-1,1-dioxide.

The crystalline modification of the acetic acid solvate of the compound 4-hydroxy-2-methyl-N-(5-methyl-2-thiazolyl)-2H-1,2-benzothiazin-3-carboxamide-1,1-dioxide according to the invention is characterised by a melting point of T_{mp.}=263±5° C. (determined by DSC=Differential Scanning Calorimetry; evaluated by the peak maximum; heating rate: 10° C./min). The value given was determined using a DSC 821^{e} made by Messrs Mettler Toledo.

A second object of the present invention is therefore the new crystalline modification of the compound 4-hydroxy-2-methyl-N-(5-methyl-2-thiazolyl)-2H-1,2-benzothiazin-3-carboxamide-1,1-dioxide in the form of an acetic acid solvate, characterised by a melting point of T_{mp.}=263±5° C. (determined by DSC; evaluated by the peak maximum; heating rate: 10° C./min).

The crystalline acetic acid solvate of the compound of 4-hydroxy-2-methyl-N-(5-methyl-2-thiazolyl)-2H-1,2-benzothiazin-3-carboxamide-1,1-dioxide according to the invention was investigated in more detail by x-ray powder diffraction. The diagram obtained is shown in FIG. 1.

Table 1 that follows contains the data obtained in this analysis:

TABLE 1
X-ray powder reflections and intensities (standardised) of
the acetic acid solvate of the compound 4-hydroxy-2-methyl-N-
(5-methyl-2-thiazolyl)-2H-1,2-benzothiazin-3-carboxamide-
1,1-dioxide.

| 2-θ | d value | intensity |
|---|---|---|
| [°] | [Å] | [%] |
| 5.23 | 16.91 | 21.1 |
| 10.44 | 8.47 | 52.6 |
| 11.19 | 7.90 | 40.0 |
| 11.52 | 7.68 | 14.5 |
| 13.15 | 6.73 | 21.6 |
| 13.60 | 6.51 | 52.9 |
| 13.95 | 6.34 | 15.4 |
| 14.67 | 6.03 | 44.4 |
| 15.34 | 5.77 | 36.4 |
| 15.48 | 5.72 | 19.2 |
| 15.71 | 5.64 | 21.2 |
| 15.81 | 5.60 | 23.2 |
| 16.62 | 5.33 | 14.5 |
| 17.62 | 5.03 | 20.4 |
| 18.45 | 4.80 | 23.8 |
| 18.72 | 4.74 | 41.9 |
| 19.70 | 4.50 | 14.4 |
| 20.57 | 4.31 | 20.6 |
| 21.01 | 4.23 | 27.4 |
| 22.06 | 4.03 | 26.4 |
| 22.22 | 34.00 | 26.4 |
| 22.51 | 3.95 | 25.6 |
| 22.71 | 3.91 | 22.9 |
| 23.24 | 3.82 | 14.6 |
| 24.20 | 3.68 | 14.8 |
| 24.85 | 3.58 | 15.4 |
| 25.20 | 3.53 | 14.6 |
| 25.99 | 3.43 | 100.0 |
| 26.72 | 3.33 | 17.9 |
| 26.88 | 3.31 | 26.0 |
| 27.40 | 3.25 | 15.1 |
| 28.13 | 3.17 | 12.7 |
| 28.79 | 3.10 | 15.1 |
| 28.90 | 3.09 | 15.1 |
| 29.46 | 3.03 | 12.8 |
| 29.92 | 2.98 | 13.6 |
| 30.66 | 2.91 | 9.9 |
| 31.28 | 2.86 | 10.5 |
| 31.53 | 2.84 | 10.3 |
| 31.74 | 2.82 | 11.1 |
| 32.05 | 2.79 | 11.3 |
| 32.37 | 2.76 | 11.6 |
| 32.90 | 2.72 | 9.5 |
| 33.55 | 2.67 | 9.0 |
| 34.41 | 2.60 | 9.5 |
| 35.05 | 2.56 | 16.6 |
| 35.38 | 2.53 | 9.1 |
| 35.59 | 2.52 | 9.4 |
| 35.80 | 2.51 | 9.4 |
| 37.00 | 2.43 | 15.1 |
| 37.17 | 2.42 | 10.9 |
| 37.47 | 2.40 | 8.3 |
| 38.00 | 2.37 | 7.1 |
| 39.26 | 2.29 | 7.2 |
| 39.53 | 2.28 | 6.8 |

In Table 1 above the value “2 Θ [°]” denotes the angle of diffraction in degrees and the value “d[Å]” denotes the specified distances in Å between the lattice planes.

The x-ray powder diagram was recorded, within the scope of the present invention, using a Bruker D8 Advanced-diffractometer fitted with a location-sensitive detector (OED) and a Cu anode as the x-ray source (CuK_{□}, radiation, □=1.5406 Å, 40 kV, 40 mA).

According to the findings shown in Table 1 the present invention relates to a new modification of the compound 4-hydroxy-2-methyl-N-(5-methyl-2-thiazolyl)-2H-1,2-benzothiazin-3-carboxamide-1,1-dioxide in the form of a crystalline acetic acid solvate, characterised in that in the x-ray powder diagram it has, inter alia, the characteristic values d=8.47 Å, 7.90 Å, 6.51 Å, 6.03, 4.74 and 3.43 Å with an intensity of more than 40%.

The meloxicam-acetic acid solvate is prepared for example by recrystallising meloxicam from glacial acetic acid.

The invention further relates to a pharmaceutical composition containing the crystalline acetic acid solvate according to the invention optionally together with one or more inert carriers and/or diluents and also to a process for preparing this pharmaceutical composition, characterised in that the acetic acid solvate according to the invention is incorporated in one or more inert carriers and/or diluents by a non-chemical method.

Experimental Section

### 4-hydroxy-2-methyl-N-(5-methyl-2-thiazolyl)-2H-1,2-benzothiazin-3-carboxamide-1,1-dioxide acetic acid solvate

29 g (83 mmol) of meloxicam (4-hydroxy-2-methyl-N-(5-methyl-2-thiazolyl)-2H-1,2-benzothiazin-3-carboxamide-1,1-dioxide) are recrystallised from 300 mL of absolute acetic acid. The precipitate is suction filtered at ambient temperature, washed with 30 mL of absolute acetic acid and dried for two hours at 30° C. in a circulating air dryer. The desired acetic acid solvate of the 4-hydroxy-2-methyl-N-(5-methyl-2-thiazolyl)-2H-1,2-benzothiazin-3-carboxamid-1,1-dioxide is obtained in a yield of 31.5 g (92% of theory) in the form of yellow crystals. The crystals contain 1 mol of acetic acid according to ¹H-NMR.

melting point: T_{mp.}=263±5° C. IR spectrum (KBr): ν=3116, 3005, 2956, 2856, 2673, 2609, 2534, 1707 cm⁻¹.

### BRIEF DESCRIPTION OF THE FIGURES

FIG. 1 shows the X-ray powder diffractogram of the crystalline acetic acid solvate of the compound 4-hydroxy-2-methyl-N-(5-methyl-2-thiazolyl)-2H-1,2-benzothiazin-3-carboxamide-1,1-dioxide.

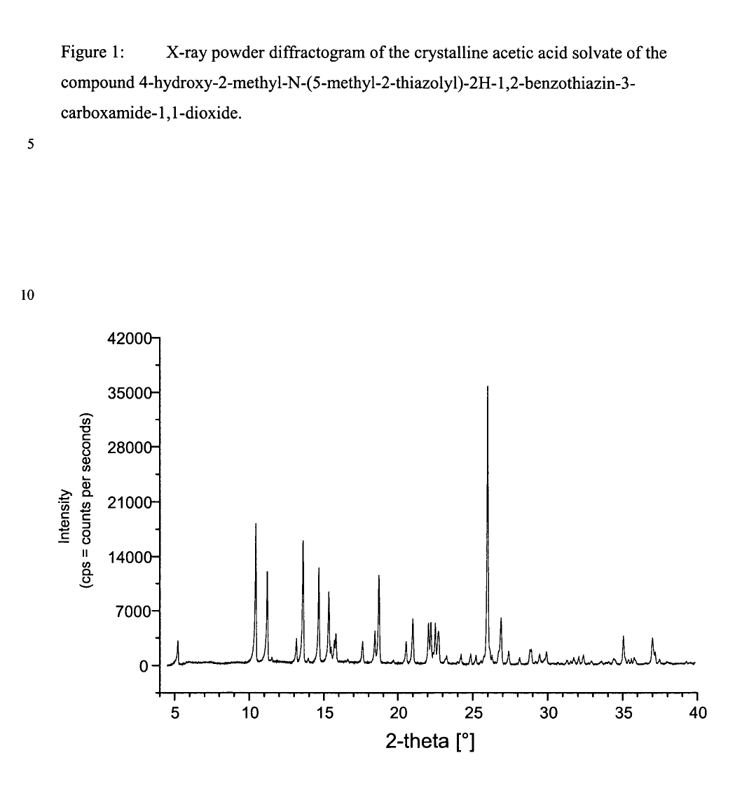

What is claimed is:

1. A crystalline acetic acid solvate of the compound 4-hydroxy-2-methyl-N-(5-methyl-2-thiazolyl)-2H-1,2-benzothiazin-3-carboxamide-1,1-dioxide (meloxicam).

2. The crystalline acetic acid solvate according to claim 1, having a melting point of T_{mp.}=263±5° C. (determined by DSC; evaluation by peak maximum; heating rate: 10° C./min).

3. The crystalline acetic acid solvate according to claim 1, the x-ray powder diagram of which has, inter alia, the characteristic values d=8.47 Å, 7.90 Å, 6.51 Å, 6.03, 4.74 and 3.43 Å with an intensity of more than 40%.

4. A process for preparing a crystalline acetic acid solvate according to claim 1 comprising recrystallizing meloxicam from acetic acid.

5. A pharmaceutical composition containing a crystalline acetic acid solvate according to claim 1 together with one or more inert carriers and/or diluents.

6. A method for treating inflammation, acute episodes of intermittent and chronic activated arthrosis, rheumatoid arthritis (chronic polyarthritis), Bechterew's disease, neoplasias, or acute and persistent pain which comprises administering to a mammal in need of such treatment a therapeutically effective amount of a crystalline acetic acid solvate in accordance with claim 1.
